(19) United States
(12) Patent Application Publication
(10) Pub. No.: US 2005/0271712 A1
(43) Pub. Date: Dec. 8, 2005

(54) Multilayer wound covering and therapeutic methods thereof

(76) Inventor: Donald T. Shannon, Trabuco Canyon, CA (US)

Correspondence Address: Manfred E. Wolff;Intellepharm, Inc., 1304 Morningside Drive, Laguna Beach, CA, 92651-2809, US

(21) Appl. No.: 10/862,167

(22) Filed: Jun. 4, 2004

(51) Int. Cl. A61K 9/127

(52) U.S. Cl. 424/445

(57) ABSTRACT

A laminar covering for human skin wounds comprising a biocompatible substantially non-porous elastomer membrane non-delaminably bonded to a biocompatible porous polymer membrane whereby the porous polymer membrane is adapted for sealable ingrowth by skin cells and can undergo mechanical separation from the ingrowth without injury to the cells. Methods for use of the device and an article of manufacture for its packaging are also taught.

### BACKGROUND ART

The present invention is related to methods and apparatus for the treatment of wounds to the skin. More specifically, this invention is related to methods and a device particularly adapted to covering of wounds and burns to the skin as a “bridge to transplant” temporary skin replacement material, whereby the use of skin transplant procedures is broadly enabled, and wherein the functional utility, ease of use, and wide applicability of the device in medical practice constitutes progress in science and the useful arts. Furthermore, the present invention teaches processes for the use of the device in medical practice.

Skin: The human skin (FIG. 1) serves the two common functions of protection from, and communication with, the environment. The uppermost layers of the skin are dead, but the underlying dermis is richly endowed with living tissue that can respond rapidly to change. A variety of nerve endings constantly report current conditions, and the body makes continuous adjustments in response. It has been said that the skin is the largest and most versatile organ of the human body. It shields against injury, it shields against foreign matter and disease organisms, and it shields against potentially harmful rays of the sun. It also regulates internal body temperature through its insulating ability and its influence on the blood flow. The skin achieves strength and pliability by being composed of a number of layers oriented so that each complements the others structurally and functionally. To allow communication with the environment, countless nerves—some of which are modified as specialized receptor end organs, and others of which are more or less structureless—come as close as possible to the surface layer; nearly every skin organ is enwrapped by skeins of fine sensory nerves.

The dermis makes up the bulk of the skin and provides physical protection. It is composed of an association of collagenous fibers with glycosaminoglycans capable of holding a large amount of water to maintain the turgidity of the skin. A dermal network of extendable elastic fibers keeps the skin taut and restores it after it has been stretched. The hair follicles and skin glands are derived from the epidermis but are deeply embedded in the dermis. The dermis is richly supplied with blood vessels, although none penetrates the living epidermis. The epidermis receives materials only by diffusion from below. The dermis also contains nerves and sense organs at various levels.

Covering the dermis is the epidermis, which is divisible into a lower layer of living cells and a superficial layer of compact dead cells. Epidermal cells, called keratinocytes, multiply chiefly at the base in contact with the dermis, forming a living Malpighian layer. These keratinocytes gradually ascend to the surface of the epidermis, manufacturing keratin, an insoluble filamentous protein composed of polypeptide chains that are stabilized by disulfide linkages. The keratinocytes finally die in the upper part, forming a horny layer.

Burns: Burns are a major and unique problem in the field of injury and surgery. Estimates for hospitalizations from burns range from 60,000-80,000 annually at a cost of $36,000-$117,000 per patient. About 10,000 persons in the United States die annually from the effects of severe burns. In time of war, these numbers are even higher. Although in the majority of other injuries or forms of surgery a person usually remains in a precarious balance between life and death for only a few days, in deep or extensive burns the victim's life remains in jeopardy for weeks. Improvements, such as the present invention, in the treatment of burns are therefore of substantial practical importance and utility.

In second degree burns (FIG. 2), damage extends through the entire epidermis and part of the dermis and is characterized by redness and blisters. The deeper the burn the more prevalent the blisters. These injuries may be extremely painful. Although most superficial second degree burns heal promptly, deep second degree burns may take up to 4-15 weeks to heal. Serious scarring, fluid losses, and metabolic disturbances may occur.

Third-degree burns (FIG. 3), otherwise termed full-thickness burns, destroy the entire thickness of the skin. The surface of the wound is leathery and may be brown, tan, black, white, or red. There is no pain, because the pain receptors have been obliterated along with the rest of the dermis. Blood vessels, sweat glands, sebaceous glands and hair follicles are all destroyed in skin that suffers a full-thickness burn. Fluid losses and metabolic disturbances associated with these injuries are grave. Treatment of deep second degree burns and third degree burns normally includes cleansing of the wound and application of antibacterials. Removal of necrotic tissues is normally undertaken within 24-48 hours of the injury. Efforts to cover the burned area with skin grafts may then be considered.

Skin grafts: A number of serious problems are associated with the utilization of skin grafts for the treatment of burns. Skin grafts may either be autografts, i.e., skin derived from another site of the patient under treatment, or allografts, i.e. skin derived from another individual or cadaver skin. Skin allografts seem to be rejected more aggressively than any other allograft tissue. With autografts, the donor skin is restricted to what the patient has available, which is often limited. Full thickness free-skin grafts produce good cosmetic results, but unless small, result in a donor site that may itself need skin grafting.

Skin grafts are divided into two major categories: full-thickness skin grafts (FTSGs) and split-thickness skin grafts (STSGs). FTSGs of about 0.48 mm thickness (deep dermis grafts) include a thick dermal layer. STSGs may be light dermis grafts (of about 0.33 mm thickness) comprising mostly epidermis plus a thin layer of dermis (FIG. 5), or thin epidermal grafts of about 0.15 mm thickness (FIG. 4). STSGs are the most common grafts in plastic surgery. They can be taken quickly from big areas to cover large defects. However, considerable time may be required for the donor sites to heal and to become eligible for further skin harvesting (reharvesting). In an effort to reduce the number autografts required, autograft skin is usually meshed and stretched widely in order to increase the area of wound coverage of a given graft. Meshing results in a four-fold or more expansion and stretching produces a two-fold expansion in the area of coverage. Such meshing and stretching can result in cosmetically undesirable scarring and marking of the healed skin, as well as in permanent contractions. The standard for rapid closure of full-thickness wounds is still a graft of split-thickness, autologous skin.

Two possible solutions to the problems in obtaining sufficient quantities of autologous skin as skin grafts comprise the use of allograft skin and the use of cultured keratinocyte grafts. The use of cultured keratinocytes in clinical practice has made it clear that stable closure of full thickness wounds needs dermis as well as epidermis. Ideally, dermis for this purpose contains living skin cells and is autologous. The closest substitute for autograft containing living cells is fresh or cryopreserved allograft skin. Dermis is a complex tissue and cannot be grown in vitro. No synthetic dermal replacement has been found to equal allograft dermis in closing wounds. Soluble proteins released by living dermal cells probably contribute to the dermo-epidermal interaction that improves grafting results.

Human cadaveric skin allograft, fresh or cryopreserved, is frequently resorted to for covering excised burn wounds, but this strategy also has severe limitations. The cryopreserved product is inferior to fresh skin, probably as a result of cellular damage sustained in the freeze-thaw process. Even the fresh product is limited in its utility owing to immunological rejection. Additionally, the use of cadaver skin raises the possibility of transmitting serious infectious materials, such as HIV, from infected grafts derived from infected cadavers. Importantly, transmission of viral, bacterial, and fungal pathogens has been reported from most types of tissue commonly transplanted. Although testing reduces the risks of transmission, safety also relies on careful selection of donors on the basis of their medical and social history. Here, the volunteer status of a cadaver tissue donor is clearly not comparable to that of a blood donor. Surgical patients who become tissue donors are actively approached pre-operatively to consider donation, as are the families of potential cadaveric donors. Additional risk arises from the inability to take first hand medical and social histories from those who donate tissues after death. Information must be gleaned from relatives, general practitioners, and pathologists, with particular emphasis on potential transmission of diseases of unknown etiology, such as malignancy. Unlike blood, many non-viable tissues can be cleared of bacteria, and possibly viruses, by exposure to ionizing radiation or ethylene oxide gas. Even minimal processing of tissues seems to reduce the risk of HIV transmission. However, when tissue viability is required this is not an option. Cadaveric allograft skin is also expensive, and is often in short supply. In the United States, an effort has been made to address this shortage through skin banking. The American Association of Tissue Banks (AATB), founded in 1976 lists 18 banks accredited to process allograft skin, mostly by cryopreservation. However, supplies are still short. In the EU the situation is substantially more severe as of 2000, owing to an inadequate number of tissue banks that offer this service.

The possibility of infection of the burn site is an increasingly great problem during the course of burn treatment. Life-threatening infections become more common, the longer the burn site is inadequately by a stable skin graft. Because of the difficulties in harvesting an adequate supply of autograft, it is clear that a suitable “bridge to transplant” temporary skin replacement material could improve the chances of burn survival, obviate the possibility of transmission of infection, and lead to a better recovery of function and appearance for other surgical procedures.

As used herein, a “bridge to transplant” is a temporary skin replacement material that becomes sealably integrated with living skin, that can be left in place for at least about eight weeks, and that can be removed without tissue damage, in order to provide time to allow autologous transplant donor site(s) to mature for re-harvesting. From the foregoing discussion, it is clear that such a “bridge to transplant” would confer important benefits on the treatment of burns in terms of reduction of adverse events related to rejection and infection.

Here, I consider possible existing technologies that could serve as a “bridge to transplant” temporary skin replacement material that becomes sealably integrated with living skin, that can be left in place for at least about eight weeks, and that can be removed without tissue damage, in order to provide time to allow autologous transplant donor site(s) to mature for re-harvesting. I exclude from consideration short-term burn dressings such as “OPSITE FLEXIGRID®” (Smith & Nephew Healthcare Ltd.) and “DUODERM®” (ConvaTec, Inc.), that differ fundamentally from the present invention, in that they can be left in place no longer than about a week, which is not long enough to provide a bridge to transplant. Likewise, I exclude from consideration skin substitutes that include living cells, as they differ fundamentally from the present invention in that the presence of the living cells results in an expensive product with either a short shelf life of a few days, or frozen storage requirements, or both. Such skin substitutes that incorporate living cells include “TRANCYTE®” (Advanced Tissue Sciences), which is similar to the product “BIOBRANE®” that is discussed below, but is seeded with neonatal fibroblasts. Likewise, “APLIGRAF® (Organogenesis Inc.), is seeded with neonatal fibroblasts and neonatal keratinocytes; “DERMAGRAFT® (Advanced Tissue Sciences, Inc.), is seeded with neonatal fibroblasts, “EPICEL®” (Genzyme Corporation), is seeded with cultured autologous keratinocytes; “VIVODERM®” (ER Squibb & Sons, Inc.), is seeded with cultured autologous keratinocytes; and, “ORCEL®” (Ortec International, Inc.) is seeded with human allogeneic epidermal keratinocytes and dermal fibroblasts.

“BIOBRANE” (Dow Hickam/Bertek Pharmaceuticals) is a bilaminate membrane consisting of nylon mesh fabric bonded to a thin layer of silicone. The nylon mesh is coated with peptides derived from porcine type I collagen, in order to aid adherence to the wound bed and fibrovascular ingrowth. It is recommended for use within the first 6 hours of injury on donor sites and superficial partial-thickness burns that are expected to heal within 14 days. The nylon mesh must be peeled away from the healing wound at some point in the treatment, which can result in renewed injury to the wound. “INTEGRA®” (Integra Life Sciences Corporation) (D. P. Orgill et al., U.S. Pat. No. 5,716,411) has an inner layer composed of collagen fibers and glycosaminoglycan. When placed on a wound where burned skin has been removed, it provides a framework for the blood vessels and dermal skin cells to grow into a new skin layer. The biodegradable collagen network obviates the need to peel the device from the wound as healing progresses. A silicone outer layer temporarily closes the wound to ward off infection and control fluid and heat loss. Difficulties have been reported in connection with the use of this product including that it is “expensive” with a “learning curve reported to be steep, with high failure rates initially” (Jones, I., Currie, L. and Martin, R. (2002) A guide to biological skin substitutes. Br J Plast Surg, 55, 185-93).

Thus, in spite of extended efforts in academic medicine and the pharmaceutical industry, there remains a need for improvement in the construction and function of devices particularly adapted to covering of wounds and burns to the skin as a “bridge to transplant” temporary skin replacement material. Even though wound coverings are used extensively in medical practice, prior devices, products, or methods available to medical practitioners have not adequately addressed the need for “bridge to transplant” temporary skin replacement materials. Thus, as pioneers and innovators attempt to make methods and apparatus particularly adapted to covering of wounds and burns to the skin, a “bridge to transplant” temporary skin replacement material that becomes sealably integrated with living skin, can be left in place for at least eight weeks, and can be removed without tissue damage in order to allow autologous transplant donor site(s) to mature for re-harvesting provides improved skin transplant procedures that are broadly enabled. The functional utility, ease of use, and wide applicability of the device of this invention in medical practice make it safer, cheaper, more universally used, and of higher quality than any other. No other device has approached these objectives in combination with simplicity and reliability of operation, until the teachings of the present invention. It is respectfully submitted that other references merely define the state of the art or show the type of systems that have been used to alternately address those issues ameliorated by the teachings of the present invention. Accordingly, further discussions of these references has been omitted at this time due to the fact that they are readily distinguishable from the instant teachings to one of skill in the art.

### OBJECTS AND SUMMARY OF THE INVENTION

Accordingly, it is an object of the present invention to provide methods and apparatus particularly adapted to covering of wounds and burns to the skin as a “bridge to transplant” temporary skin replacement material that becomes sealably integrated with living skin, can be left in place for at least eight weeks, and can be removed without tissue damage in order to allow autologous transplant donor site(s) to mature for re-harvesting. A further object of the present invention is to provide a “bridge to transplant” temporary skin replacement material that can be peeled away from the healing wound at some point in the treatment without renewed injury to the wound. Another object of the present invention to provide a “bridge to transplant” temporary skin replacement material that temporarily closes the wound to ward off infection. Still another object of the present invention is to provide a “bridge to transplant” temporary skin replacement material that is comparatively inexpensive. An additional object of the present invention is to provide a “bridge to transplant” temporary skin replacement material that has a learning curve that is not steep. Even still a further object of the present invention is to provide a “bridge to transplant” temporary skin replacement material that does not have high failure rates initially. Yet still a further object of this invention is to provide methods and apparatus that are suitable for use with a variety of polymeric materials. Even still another object of this invention is to provide a “bridge to transplant” temporary skin replacement material that temporarily closes the wound to control fluid loss. Even yet still another object of this invention is to provide a “bridge to transplant” temporary skin replacement material that temporarily closes the wound to control heat loss. Even an additional object of this invention is to provide an article of manufacture for packaging the apparatus of the invention. Even still an additional object of this invention is to provide a device capable of delivering an antimicrobial formulation to the wound.

These and other objects are accomplished by the parts, constructions, arrangements, combinations and subcombinations comprising the present invention, the nature of which is set forth in the following general statement, and preferred embodiments of which—illustrative of the best modes in which applicant has contemplated applying the principles—are set forth in the following description and illustrated in the accompanying drawings, and are particularly and distinctly pointed out and set forth in the appended claims forming a part hereof.

### BRIEF EXPLANATION OF THE DRAWINGS

The foregoing and other objects and advantages of the invention will be appreciated more fully from the following further description thereof, with reference to the accompanying drawings in which like parts are given like reference numerals and wherein:

FIG. 1 is a schematic rendering of an enlarged cross sectional view of the skin of a human patient.

FIG. 2 is a schematic rendering of an enlarged cross sectional view of the skin of a human patient, a portion of which has suffered a second degree burn, wherein the burned area has been debrided to remove damaged tissue.

FIG. 3 is a schematic rendering of an enlarged cross sectional view of the skin of a human patient, a portion of which has suffered a third degree burn, wherein the burned area has been debrided to remove damaged tissue.

FIG. 4 is a schematic rendering of an enlarged cross sectional view of a split-thickness skin graft (STSG) comprising a thin epidermal grafts of human skin.

FIG. 5 is a schematic rendering of an enlarged cross sectional view of a split-thickness skin graft (STSG) comprising a light dermis graft of mostly epidermis plus a thin layer of dermis of human skin.

FIG. 6 is a schematic rendering of an enlarged cross sectional view of a “bridge to transplant” temporary skin replacement material of the present invention having two layers.

FIG. 7 is a schematic rendering of an enlarged cross sectional view of a “bridge to transplant” temporary skin replacement material of the present invention having three layers.

FIG. 8 is a diagrammatic view of an article of manufacture, comprising packaging material, the “bridge to transplant” temporary skin replacement material of the present invention, and a label.

### DETAILED DESCRIPTION OF THE PREFERRED EMBODIMENTS

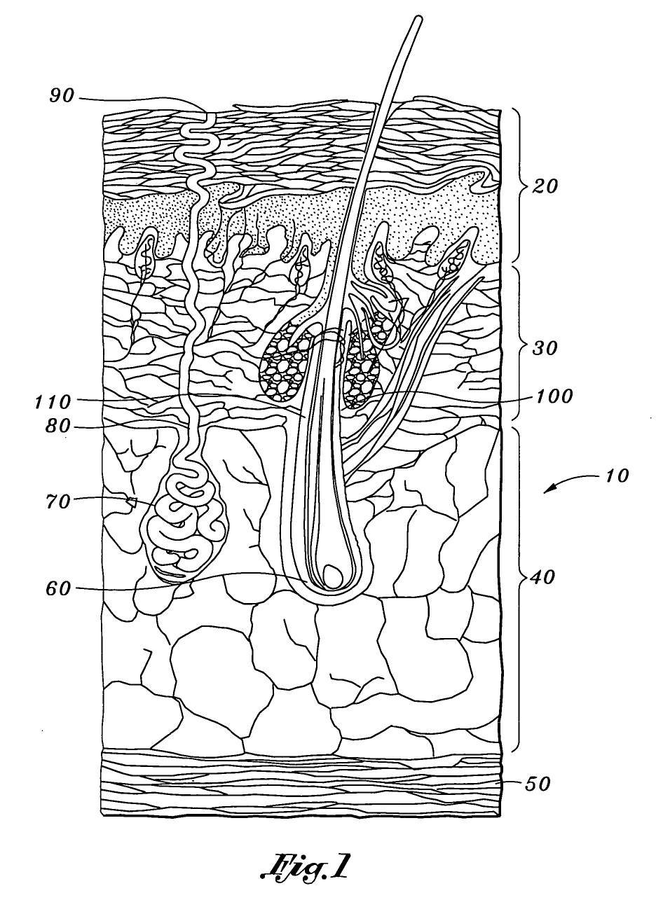

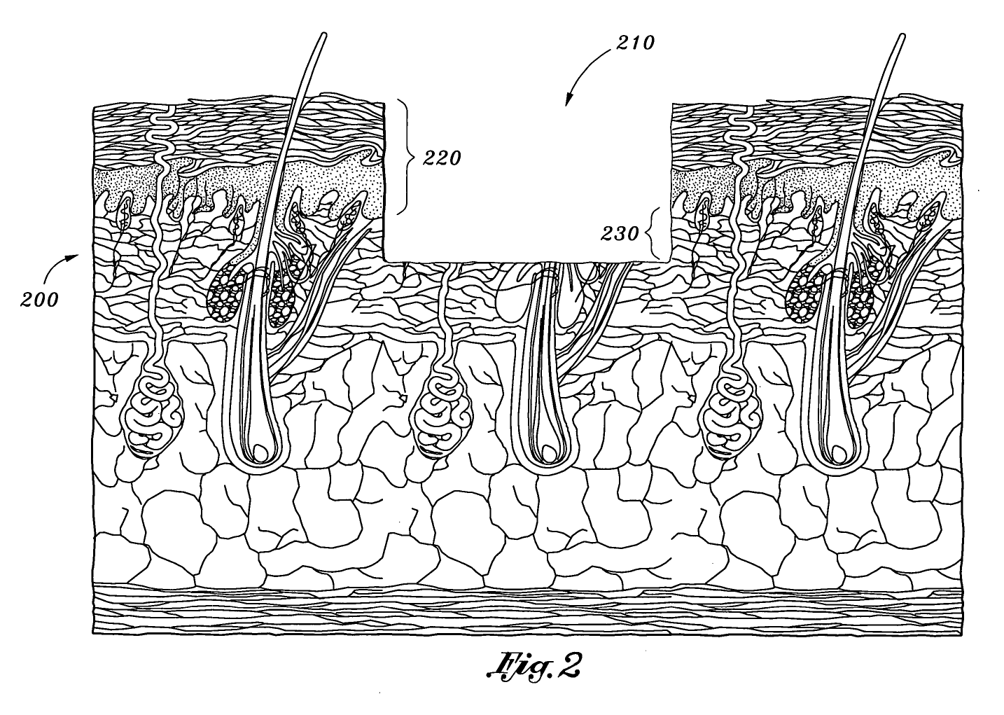

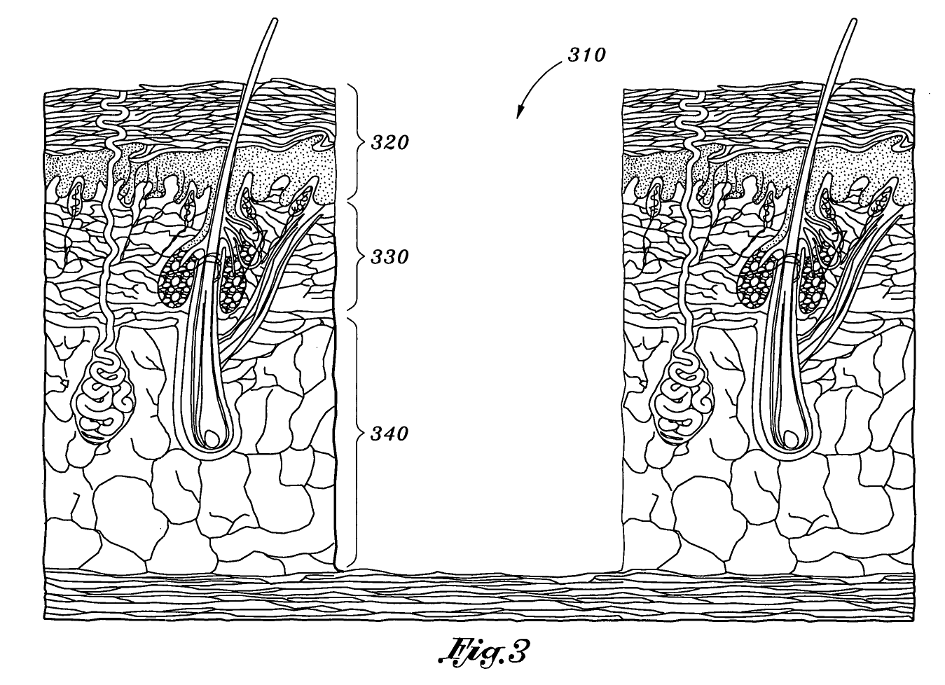

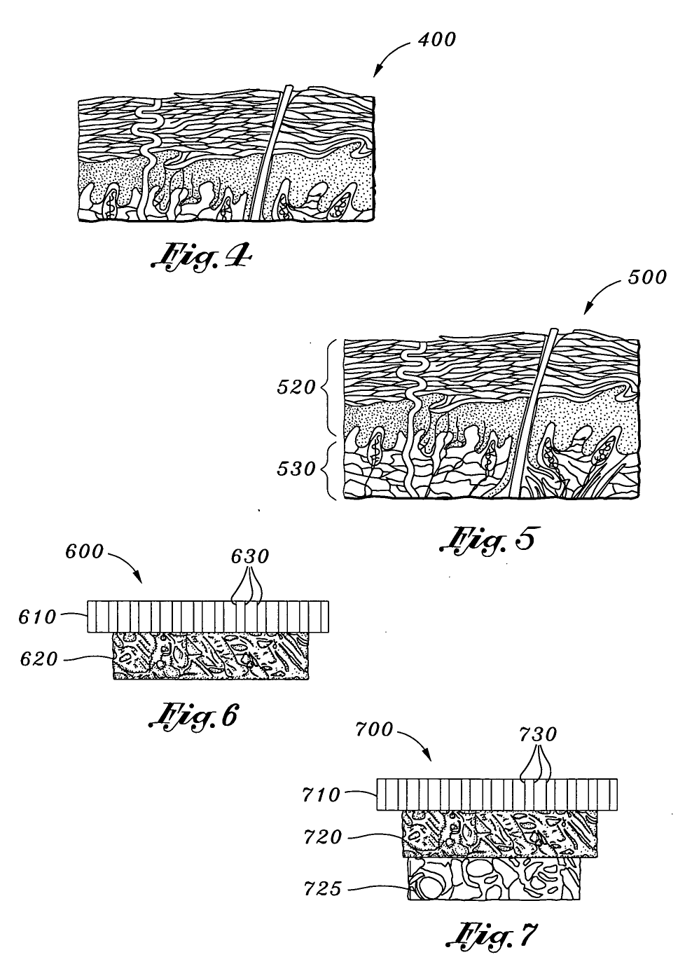

With reference to FIG. 1 a schematic rendering of an enlarged cross sectional view of normal human skin is shown and generally indicated at 10. The epidermis 20 overlies the dermis 30. Dermis 30 overlies the subcutaneous connective tissue 40. Underlying tissue 40 is the muscle layer 50. A hair papilla 60 and a sweat gland 70 are located in tissue 40. Gland 70 extends through a sweat gland duct 80 and exits epidermis 20 through a sweat gland pore 90. Sebaceous glands 100 lubricate hair follicle 110. With reference to FIG. 2 a schematic rendering of an enlarged cross sectional view of human skin after debridement of a second degree burn is shown and generally indicated at 200. The area of wound debridement is generally indicated at 210, and includes the full thickness of wounded epidermis 220 the partial thickness of wounded dermis 230. With reference to FIG. 3 a schematic rendering of an enlarged cross sectional view of human skin after debridement of a third degree burn is shown and generally indicated at 300. The area of wound debridement is generally indicated at 310, and includes the full thickness of wounded epidermis 320, the full thickness of wounded dermis 330, and the underlying connective tissue 340. With reference to FIG. 4 a schematic rendering of an enlarged cross sectional view of a split-thickness skin graft (STSG) comprising a thin epidermal graft of human skin is shown and generally indicated at 400. With reference to FIG. 5 a schematic rendering of an enlarged cross sectional view of a split-thickness skin graft (STSG) comprising a light dermis graft of mostly epidermis plus a thin layer of dermis of human skin is shown and generally indicated at 500.

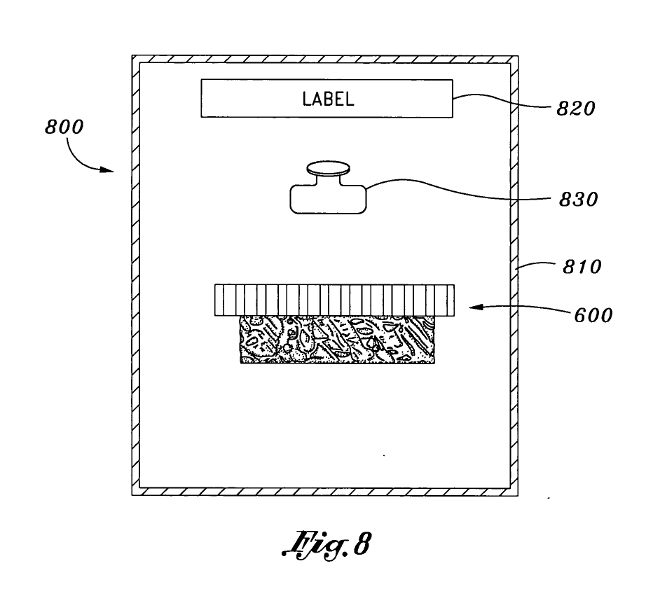

With reference to FIG. 6 a schematic rendering of an enlarged cross sectional view of a “bridge to transplant” temporary skin replacement material of the present invention is shown and generally indicated at 600. Material 600 comprises a flexible, porous biocompatible membrane 620 adapted for epidermal ingrowth to which a non-porous thermoplastic biocompatible elastomer 610, perforated by a multiplicity of laser induced ablations 630, is substantially non-delaminably bonded by known methods, for example by thermal bonding. With reference to FIG. 7 a schematic rendering of an enlarged cross sectional view of a “bridge to transplant” temporary skin replacement material of the present invention is shown and generally indicated at 700. Material 700 comprises a flexible, porous biocompatible fluoropolymer membrane 725 adapted for dermal ingrowth to which a flexible, porous biocompatible membrane 720 adapted for epidermal ingrowth is substantially non-delaminably bonded by known methods, for example by thermal bonding. A flexible non-porous thermoplastic biocompatible elastomer 710, perforated by a multiplicity of laser induced ablations 730, is substantially non-delaminably bonded to membrane 720 by known methods, for example by thermal bonding. With reference to FIG. 8 a diagrammatic view of an article of manufacture generally indicated at 800, comprising packaging material 810, a “bridge to transplant” temporary skin replacement material of the present invention 600, a label 820 and a container 830 of a pharmaceutically acceptable topical antimicrobial formulation is shown.

A crucially important aspect of this invention is the interaction between the living dermis and/or epidermis of the patient and the porous membranes of the invention. Thus, in order to provide a “bridge to transplant” that can remain in place for an extended period, it is necessary that the device of the invention be sealably integrated with living tissue, and able to be removed without tissue damage. Although conventional wound coverings have long used adhesive means adapted for sealing such coverings to the skin, such means are not adequate for the purposes of the present invention. Such adhesive means adhere to the dead cornified layer of the epidermis, which is sloughed off In the course of 1-2 weeks. Consequently, this is wholly inadequate for a “bridge to transplant.” Yet a sealable integration, capable of excluding contamination of the wound by infectious organisms, is required for the laminar covering for human skin wounds to be left in place for an extended period. Furthermore, a second requirement for such a seal is that the sealed covering can be removed from the wound without damage to the tissue. The first requirement—that of the tight seal that is impassable by infectious organisms—can be met, for example, by ingrowth of skin cells into pores of porous biocompatible membrane 720 or 725 of the invention. The importance of the specific surface (cm²/g) as a function of pore size in this connection has been noted by Yannas et al. (Yannas, I. V., Lee, E., Orgill, D. P., Skrabut, E. M. and Murphy, G. F. (1989) Synthesis and characterization of a model extracellular matrix that induces partial regeneration of adult mammalian skin. Proc Natl Acad Sci USA, 86, 933-7). In my opinion, it is reasonable to believe that the specific limits on the mean pore diameter that govern the sealability of the membrane suggest that an ingrowth of tissue into the pores is required for sealability to be achieved. However, the formation of a sealable union of this type is not the only requirement for the “bridge to transplant”, inasmuch as removal of the device is necessary before transplantation can be undertaken. Here, the seal must be breakable without injuring the sealing tissue. In my invention, I find that a “non-stick” surface satisfies these requirements for the porous membrane(s) of the device.

The degree of adhesion of biological materials to surfaces of artificial substrates is known to vary depending upon the critical surface tension of the substrate, the chemical constitution of the substrate, and the chemical constitution of the biological material. Low critical surface tension substrates discourage such adhesion in comparison to higher critical surface tension substrates. For example, PMMA or polymethylmethacrylate substrates have a high critical surface tension that promotes the adhesion of biological materials markedly. Conversely, polytetrafluoroethylene has a low critical surface tension and is known for its “non-stick” qualities. In my invention, I find that a CST (critical surface tension) value up to 29 dynes/cm as measured from the shapes of simple liquids in contact with the surface, is a characteristic of materials that behave as essentially nonstick surfaces that satisfy these requirements for the porous membrane(s) of the device.

Critical Surface Tension*

| | | Critical surface |
|---|---|---|
| | | tension |
| Material | Product | (dynes/cm) |
| Polymers | | |
| Polyetherurethane | Pellethane 80A | 19.3 |
| Polyetherurethane urea | Biomer | 23.0 |
| Polyethylene | | 31-33 |
| Poly(methyl methacrylate) | | 39.0 |
| Poly(tetrafluoroethylene) | | 18 |
| Polyvinylchloride | | 41 |

*The critical surface tension is the surface tension of a liquid that would completely wet the solid of interest.

Thus, my invention comprises a laminar covering for human skin wounds comprising a biocompatible substantially non-porous elastomer membrane; and a first biocompatible porous polymer membrane non-delaminably bonded to the non-porous elastomer membrane wherein the first porous polymer membrane has a critical surface tension of less than about 29 dynes per centimeter, whereby the first porous polymer membrane is adapted for sealable ingrowth by skin cells; and the membrane can undergo mechanical separation from the ingrowth without injury to the cells. The covering can be left in place for at least about eight weeks between the time of the ingrowth and the separation. The covering is useful when employed in a method for the treatment of burns, comprising applying the covering to a patient in need of such treatment wherein the application is effective to ameliorate one or more of the symptoms of the burns. The first porous polymer membrane may be penetrated by a multiplicity of perforations that may have a diameter between at least about 5μ and about 100μ. The substantially non-porous elastomer membrane may have a thickness between at least about 2.5μ and about 500μ. The pores in the first porous polymer membrane may have a mean diameter between at least about 20μ and about 125μ. The first porous polymer membrane may have a thickness between at least about 25μ and about 3000μ. The substantially non-porous elastomer membrane may be vinylidene polymer plastics, polyethylene, polypropylene, polyesters, polyamides, polyethylene terephthalate, high density polyethylene, irradiated polyethylene, polycarbonates, polyurethanes, polyvinyl chloride, polyester copolymers, polyolefin copolymers, PFA (perfluoroalkoxy), PPS, PVDF (polyvinylidene fluoride), PEEK, PS/PES, PCTFE, or PTFE. The first porous polymer membrane may be a fluorocarbon polymer like PTFE, ePTFE, FEP, PFA, PVDF, PCTFE, or ETFE. The pores of the first porous polymer membrane may contain a pharmaceutically acceptable topical antimicrobial formulation for combating infection that may include one or more of the following substances: polymyxin B, neomycin, mupirocin, amphotericin B, nystatin, norfloxacin, and ciprofloxacin. The covering may be contained in packaging material that may include a container of a pharmaceutically acceptable topical antimicrobial formulation and a label that indicates that the covering is effective for treatment comprising application to a patient afflicted with burns.

The covering may further comprise a second porous polymer membrane having a critical surface tension of less than about 29 dynes per centimeter, wherein the second porous polymer membrane is non-delaminably bonded to the first porous polymer membrane, whereby the first second polymer membrane is adapted for sealable ingrowth by skin cells, can undergo mechanical separation from the ingrowth without injury to the cells, and can be left in place for at least about eight weeks between the time of the ingrowth and the separation. The second porous polymer membrane can be a fluorocarbon polymer membrane such as porous PTFE, ePTFE, FEP, PFA, PVDF, PCTFE, and ETFE. The second porous polymer membrane can have a thickness between at least about 200μ and about 3000μ. The pores in the second porous polymer membrane can have a mean diameter between at least about 20μ and about 125μ. The pores of the second porous fluorocarbon polymer membrane may contain a pharmaceutically acceptable topical antimicrobial formulation for combating infection that may include one or more of the following substances: polymyxin B, neomycin, mupirocin, amphotericin B, nystatin, norfloxacin, and ciprofloxacin.

Thus it will be appreciated that the invention provides a new and improved covering for wounds and burns to the skin. It should be understood, however, that the foregoing description of the invention is intended merely to be illustrative thereof and that other modifications in embodiments may be apparent to those skilled in the art without departing from its spirit. On this basis, the instant invention should be recognized as constituting progress in science and the useful arts, and as solving the problems in dermatology and medicine enumerated above. In the foregoing description, certain terms have been used for brevity, clearness and understanding, but no unnecessary limitation is to be implied therefrom beyond the requirements of the prior art, because such words are used for descriptive purposes herein and are intended to be broadly construed.

Having described preferred embodiments of the invention with reference to the accompanying drawings, it is to be understood that the invention is not limited to those precise embodiments, and that the various changes and modifications may be effected therein by one skilled in the art without departing from the scope or spirit of the invention as defined in the appended claims. Thus, the scope of the invention should be determined by the appended claims and their legal equivalents, rather than by the examples given. All changes that come within the meaning and range of equivalency of the claims are to be embraced within their scope.

### DEFINITIONS

Unless defined otherwise, all technical and scientific terms used herein have the same meaning as is commonly understood by one of skill in the art to which this invention belongs. All patents and publications referred to herein are incorporated in their entirety by reference.

All abbreviations for fluorocarbon polymers used herein have the same meaning as is commonly understood by one of skill in the art to which this invention belongs. For example, PTFE refers to polytetrafluoroethylene, and ePTFE refers to expanded polytetrafluoroethylene. As further examples, FEP refers to poly(tetrafluoroethylene-co-hexafluoropropylene, PFA refers to perfluoroalkoxyalkene copolymer, PVDF refers to polyvinylidene fluoride, PCTFE refers to polychlorotrifluoroethylene, and ETFE refers to ethylene tetrafluoroethylene.

All terms for polymers used herein have the same meaning as is commonly understood by one of skill in the art to which this invention belongs. As an example, the terms “resin”, “polymer”, and “elastomer” may be used synonymously by one of skill in the art to which this invention belongs.

As used herein, “biocompatible” means not having toxic or injurious effects on biological function in a host.

As used herein, a bilaminate structure is a structure comprising two layers.

As used herein, a trilaminate structure is a structure comprising three layers.

As used herein, a non-delaminable structure is a structure comprising at least two layers wherein the layers cannot be pulled apart or separated from each other without destroying the structural integrity of the individual layers.

As used herein, the terms “sealable” and “sealably” refer to a seal sufficiently tight to block the passage of infectious organisms.

As used herein, the critical surface tension of a solid material is the surface tension of a liquid that would completely wet the solid material.

As used herein, the average pore diameter in a sample of porous polymer is the average value, expressed in μ, that is obtained using an electron microscope according to the method of Dagalakis et al., (Dagalakis, N., Flink, J., Stasikelis, P., Burke, J. F. and Yannas, I. V. (1980) Design of an artificial skin. Part III. Control of pore structure. J Biomed Mater Res, 14, 511-28).

As used herein, the skin is the membranous, protective covering of the human body consisting of epidermis and dermis.

As used herein, the terms debridement, debrided, and the like refer to the excision of devitalized tissue and foreign matter from a wound.

1. A laminar covering for human skin wounds comprising, in combination:
a biocompatible substantially non-porous elastomer membrane; and,
a first biocompatible porous polymer membrane non-delaminably bonded to said non-porous elastomer membrane;
wherein said first porous polymer membrane has a critical surface tension of less than about 29 dynes per centimeter;
whereby said first porous polymer membrane is adapted for sealable ingrowth by skin cells; and, said membrane can undergo mechanical separation from said ingrowth without injury to said cells.

2. The covering of claim 1, wherein said covering can be left in place for at least about eight weeks between the time of said ingrowth and said separation.

3. The covering of claim 1, wherein said first porous polymer membrane is penetrated by a multiplicity of perforations.

4. The covering of claim 3, wherein said perforations in said substantially non-porous elastomer membrane have a diameter between at least about 5μ and about 100μ.

5. The covering of claim 1, wherein said substantially non-porous elastomer membrane has a thickness between at least about 2.5μ and about 500μ.

6. The covering of claim 1, wherein said substantially non-porous elastomer membrane is selected from the group consisting of vinylidene polymer plastics, polyethylene, polypropylene, polyesters, polyamides, polyethylene terephthalate, high density polyethylene, irradiated polyethylene, polycarbonates, polyurethanes, polyvinyl chloride, polyester copolymers, polyolefin copolymers, PFA (perfluoroalkoxy), PPS, PVDF (polyvinylidene fluoride), PEEK, PS/PES, PCTFE, and PTFE.

7. The covering of claim 1, wherein said first porous polymer membrane is a fluorocarbon polymer membrane selected from the group consisting of porous PTFE, ePTFE, FEP, PFA, PVDF, PCTFE, and ETFE.

8. The covering of claim 1, wherein the pores in said first porous polymer membrane have a mean diameter between at least about 20μ and about 125μ.

9. The covering of claim 1, wherein said first porous polymer membrane has a thickness between at least about 25μ and about 3000μ.

10. The covering of claim 1, wherein said first porous polymer membrane is saturated with a pharmaceutically acceptable topical antimicrobial formulation.

11. The covering of claim 10, wherein said formulation includes one or more of the following substances: polymyxin B, neomycin, mupirocin, amphotericin B, nystatin, norfloxacin, and ciprofloxacin.

12. The covering of claim 1, further comprising a second porous polymer membrane having a critical surface tension of less than about 29 dynes per centimeter, wherein said second porous polymer membrane is non-delaminably bonded to said first porous polymer membrane, whereby said first second polymer membrane is adapted for sealable ingrowth by skin cells; and, said second polymer membrane can undergo mechanical separation from said ingrowth without injury to said cells.

13. The covering of claim 12, wherein said second porous polymer membrane can be left in place for at least about eight weeks between the time of said ingrowth and said separation.

14. The covering of claim 12, wherein said second porous polymer membrane is a fluorocarbon polymer membrane selected from the group consisting of porous PTFE, ePTFE, FEP, PFA, PVDF, PCTFE, and ETFE.

15. The covering of claim 12, wherein said second porous polymer membrane has a thickness between at least about 200μ and about 3000μ.

16. The covering of claim 12, wherein the pores in said second porous polymer membrane have a mean diameter between at least about 20μ and about 125μ.

17. The covering of claim 12, wherein the pores of said second porous fluorocarbon polymer membrane contain a pharmaceutically acceptable topical antimicrobial formulation.

18. The covering of claim 17, wherein said formulation includes one or more of the following substances: polymyxin B, neomycin, mupirocin, amphotericin B, nystatin, norfloxacin, and ciprofloxacin.

19. A method for the treatment of burns, comprising applying the covering of claim 1 in a patient in need of such treatment wherein said application is effective to ameliorate one or more of the symptoms of said burns.

20. A method for the treatment of burns, comprising applying the covering of claim 12 in a patient in need of such treatment wherein said application is effective to ameliorate one or more of the symptoms of said burns.

21. An article of manufacture, comprising packaging material and the covering of claim 1 contained within the packaging material, wherein said covering is effective for application to a patient afflicted with burns, and the packaging material includes a label that indicates that said covering is effective for said application.

22. An article of manufacture, comprising packaging material and the covering of claim 12 contained within the packaging material, wherein said covering is effective for application to a patient afflicted with burns, and the packaging material includes a label that indicates that said covering is effective for said application.

23. The article of manufacture of claim 21, further comprising a container of a pharmaceutically acceptable topical antimicrobial formulation.

24. The article of manufacture of claim 22, further comprising a container of a pharmaceutically acceptable topical antimicrobial.
